(19) United States
(12) Patent Application Publication
(10) Pub. No.: US 2010/0193349 A1
(43) Pub. Date: Aug. 5, 2010

(54) Ultrasonic Horn

(76) Inventor: Erik Braam, Westborough, MA (US)

Correspondence Address: K&L Gates LLP, STATE STREET FINANCIAL CENTER, One Lincoln Street, BOSTON, MA, 02111-2950, US

(21) Appl. No.: 12/696,556

(22) Filed: Jan. 29, 2010

Related U.S. Application Data
(60) Provisional application No. 61/148,531, filed on Jan. 30, 2009.

(51) Int. Cl. B01F 11/02; B06B 1/00; C07C 6/00

(52) U.S. Cl. 204/157.15; 366/116; 422/128

(57) ABSTRACT

The present teachings relate to ultrasonic horns that have improved stress profiles durability. In various embodiments, the ultrasonic horns can have geometric features designed to distribute stress more evenly along their longitudinal length.

### CROSS-REFERENCE TO RELATED APPLICATIONS

This application claims priority to and the benefit of U.S. Provisional Patent Application Ser. No. 61/148,531, filed on Jan. 30, 2009, the disclosure of which is incorporated by reference herein in its entirety.

### BACKGROUND

Ultrasonic liquid processing uses high frequency energy to cause vibration in liquids to produce physical or chemical effects. Ultrasonic waves, which typically refer to the part of the sonic spectrum that ranges from 15 kHz to 300 MHz, can be generated by an ultrasonic transducer that converts mechanical, electrical, or some other form of energy into high frequency acoustical (sound) energy. The sound energy then can be transmitted to an ultrasonic horn for amplification, and the ultrasonic horn in turn can transmit the amplified energy as high frequency vibrations to the liquid medium.

Ultrasonic horns are designed to impart energy at a selected frequency and wavelength, and to provide a selected gain. The gain of an ultrasonic horn refers to the ratio of its output amplitude to its input amplitude, where amplitude refers to the maximum displacement of a surface (e.g., the output surface and the input surface) upon excitation. Generally, a greater reduction between the input surface area and the output surface area translates to a larger gain. In other words, a large gain often is achieved at the expense of a small working surface area.

When a longitudinal horn, i.e., a horn having a longitudinal length substantially larger than its diameter, is driven at its natural (or resonant) frequency, the horn ends will expand and contract longitudinally about its node points thereby alternately lengthening and shortening portions of the horn. A longitudinal horn typically has a length that is equal to or approximates an integer multiple of one-half of the wavelength that corresponds to its resonant frequency. Depending on its length, an ultrasonic horn can have one or more nodal planes where zero amplitude of vibration is observed.

The four most common designs for longitudinal horns are step horns, conical horns, exponential horns, and catenoidal horns. Step horns usually have two or more sections, where each section has a uniform cross-sectional area but the cross-sectional areas of different sections generally decrease towards the output end. While step horns can provide a very high gain, the stress in the nodal region (which includes the nodal plane) is also highest compared to other designs when the horns are used at comparable output amplitudes. Conical horns have a linear taper from the input end to the output end. Both exponential horns and catenoidal horns have a non-linear taper, with the taper following an exponential curve and a catenoidal curve, respectively.

Despite the variety of existing horn designs, few horns can be operated at high amplitudes for an extended period without fracturing. Accordingly, there is a need in the art for ultrasonic horns than can withstand high stress for a longer period of use than existing horns.

### SUMMARY

In light of the foregoing, the present teachings provide ultrasonic horns that can provide improved stress profiles and durability. Unlike conventional horn designs, the present ultrasonic horns do not follow an overall taper from the input end to the output end. Instead, the present ultrasonic horns are characterized by a freeform shape including one or more curvilinear sections which can give rise to various advantages including minimizing local excessive stress. As a result, the present ultrasonic horns can have improved mechanical properties and can be used in high-amplitude applications for an extended time without fracturing.

An ultrasonic horn according to the present teachings generally has a solid elongated body which is adapted to transmit and amplify ultrasonic vibrations from an input end to an output end. The input end is adapted to be coupled to a source of ultrasonic vibrations (e.g., an ultrasonic transducer), while the output end is adapted to make contact with a target medium for receiving the amplified ultrasonic vibrations. The input end typically has a flat input surface but can have other shapes to facilitate coupling with the transducer. The output end can have a flat output surface or a non-planar (e.g., convex) output surface.

The solid elongated body generally has a longitudinal length extending between the input end and the output end that approximates an integer multiple of one-half of a wavelength (λ) of a resonant frequency of the material from which the solid elongated body is made. In certain embodiments, the solid elongated body can be axially symmetrical and can have a curvilinear longitudinal profile. The solid elongated body can include a plurality of transverse sections along its longitudinal length, where the cross-sectional areas within each transverse section can vary. These transverse sections can have geometrically similar (e.g., circular) cross-sections. Among these transverse sections, there can be a first transverse section that has a substantially convex longitudinal profile. This first transverse section can have a maximum diameter that is substantially the same as or greater than the diameter of the input surface. For example, the first transverse section can have a maximum diameter between about λ/4 and about λ/9, whereas the input surface can have a diameter between about λ/4 and about λ/12. The maximum diameter of the first transverse section can be located between the output end and a halfpoint of the longitudinal length. In various embodiments, the present ultrasonic horns can include a second transverse section that has a substantially concave longitudinal profile. This second transverse section can have a minimum diameter that is substantially the same as or less than the diameter of the output surface. For example, the second transverse section can have a minimum diameter between about λ/8 and about λ/40, whereas the output surface can have a diameter between about λ/6 and about λ/30. The minimum diameter of the second transverse section can be located between the input end and a halfpoint of the longitudinal length.

In various embodiments, the solid elongated body can be constructed from a unitary piece of material. In some embodiments, the material can be a corrosive-resistant metal or alloy. For example, the material can be a nickel-chromium alloy comprising titanium and aluminum.

Various embodiments of the present ultrasonic horns can have excellent stress properties. For example, the present ultrasonic horns can be configured to exhibit a maximum von Mises stress lower than about 450 MPa, lower than about 400 MPa, and lower than about 350 MPa, per 100 micron (μm) of displacement at the output end, as demonstrated in the examples hereinbelow using ultrasonic horns according to the present teachings that are composed of NIMONIC 80A™ and designed with a natural frequency of about 18 kHz. NIMONIC 80A™ is an alloy characterized by an elastic modulus of about 220 GPa, and a density of about 8100 kg/m³. Ultrasonic horns with comparable stress properties can be obtained according to the present teachings using materials that are characterized by similar physical properties.

In various embodiments, the present ultrasonic horns can be configured to reach a maximum amplitude at the output end, and provide a gain of greater than about 7.0 when in use. In certain embodiments, the output end can be configured to reach a maximum displacement amplitude of up to about 150 μm. Certain embodiments of the present ultrasonic horns can be configured to exhibit, at a halfpoint along the longitudinal length, a displacement amplitude that is equivalent to about 50-80% of the maximum displacement amplitude located at the output end.

The present teachings also relate to apparatus and systems that include the ultrasonic horns described herein, as well as processes of treating a liquid composition by ultrasonic energy with the use of the present ultrasonic horns and related apparatus and systems.

These and other objects, advantages, and features of the invention will be apparent from the description that follows.

### BRIEF DESCRIPTION OF THE DRAWINGS

It should be understood that the drawings are not necessarily to scale, with emphasis generally being placed upon illustrating the principles of the present teachings. The drawings are not intended to limit the scope of the present teachings in any way.

FIG. 1 is a side view of an ultrasonic horn in accordance with an embodiment of the present teachings.

FIG. 2 is a side view of an ultrasonic horn in accordance with another embodiment of the present teachings.

FIG. 3 illustrates two ultrasonic horns according to the present teachings, where one of them has a planar output surface, and the other has a rounded (dome-shaped) output end.

FIG. 4 is a cross-section view of a flow-through reactor that incorporates an ultrasonic horn according to the present teachings.

FIG. 5 compares the stress profiles and the displacement profiles of two embodiments of the ultrasonic horns according to the present teachings as measured along their longitudinal lengths.

### DETAILED DESCRIPTION

Throughout the application, where apparatus or compositions are described as having, including, or comprising specific components, or where processes are described as having, including, or comprising specific process steps, it is contemplated that devices or compositions of the present teachings also consist essentially of, or consist of, the recited components, and that the processes of the present teachings also consist essentially of, or consist of, the recited process steps. It should be understood that the order of steps or order for performing certain actions is immaterial so long as the method remains operable. Moreover, two or more steps or actions can be conducted simultaneously.

In the application, where an element or component is said to be included in and/or selected from a list of recited elements or components, it should be understood that the element or component can be any one of the recited elements or components, or the element or component can be selected from two or more of the recited elements or components. Further, it should be understood that elements and/or features of a composition, an apparatus, or a method described herein can be combined in a variety of ways without departing from the spirit and scope of the present teachings, whether explicit or implicit herein.

The use of the terms “include,” “includes”, “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

It has been found that ultrasonic horns having certain geometric features can have better stress tolerance (e.g., against fracturing) and in turn a longer useful life while providing a desirable gain and working surface area. More specifically, the present ultrasonic horns are adapted to provide smoother (e.g., less abrupt changes) and broader (e.g., more evenly distributed) stress profiles compared to existing ultrasonic horns. In addition, various embodiments of the present ultrasonic horns can provide a gain of greater than about 7.0, for example, greater than about 7.5, greater than about 8.0, greater than about 8.5, and greater than about 9.0, without considerably sacrificing the working surface area. Accordingly, ultrasonic horns of the present teachings can be suitable for high-amplitude, high-intensity applications as described herein.

The present ultrasonic horns are elongated bodies having a longitudinal axis, and generally are symmetrically shaped about the longitudinal axis. In various embodiments, the present ultrasonic horns have cross-sectional areas in planes normal to the longitudinal axis that vary along the longitudinal axis. The cross-sections can take various geometric shapes including circles, ovals, squares, and rectangles. In most embodiments, the ultrasonic horns according to the present teachings have circular cross-sections, making them bodies of rotation.

The present ultrasonic horns are adapted to transmit ultrasonic vibrations primarily along the longitudinal axis from an input (proximal) end to an output (distal) end. The input end typically has a flat input surface but can be non-planar to facilitate coupling with the source of ultrasonic vibrations (e.g., an ultrasonic transducer). Similarly, the output end can have a flat output surface or a non-planar output surface. In some embodiments, the output end can have a rounded tip that defines a dome-shaped output surface. For example, the rounded tip can have a spherically convex shape, and can have a maximum height that is the radius of the output end.

The output end typically has a smaller surface area than the input end. However, to provide an optimized balance between gain and working surface area, the ratio of the diameter of the output surface to the diameter of the input surface can be at least about ⅓ or greater in various embodiments. For example, the ratio of the diameter of the output surface to the diameter of the input surface (Dₒ/Dᵢ) can be greater than 0.35. In certain embodiments, Dₒ/Dᵢ can be greater than about 0.40, greater than about 0.45, greater than about 0.50, greater than about 0.55, greater than about 0.60, or greater than about 0.67. The present ultrasonic horns can have an input surface having a diameter between about λ/4 and about λ/12, and an output surface having a diameter between about λ/6 and about λ/30. In certain embodiments, Dₒ can be greater than or equal to about 40 mm.

While most prior art longitudinal horns include cross sections that generally decrease in diameter, i.e., taper, from the input end to the output end, and take the shape of a conventional step horn, conical horn, exponential horn, or catenoidal horn, the present ultrasonic horns have a freeform, curvilinear profile. More specifically, along the longitudinal axis, the present ultrasonic horns can be divided into a plurality of transverse sections having varying cross-sectional areas. For example, the present ultrasonic horns can have one or more transverse sections having increasing (e.g., non-linearly increasing) cross-sectional areas from the input end to the output end. In addition, there can be one or more transverse sections having non-linearly, non-exponentially, or non-catenoidally decreasing cross-sectional areas from the input end to the output end.

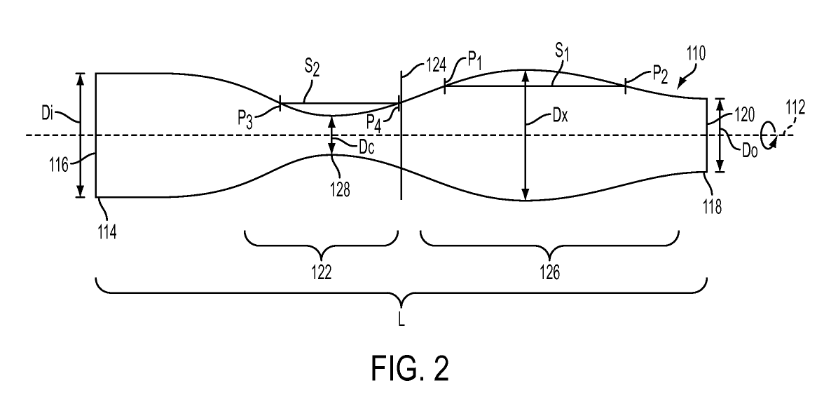

In various embodiments, the present ultrasonic horns can have at least one convex (bulbous-shaped) transverse section. A convex transverse section can be defined by a thick intermediate section between two narrower ends. Or described differently, as shown in FIG. 2, if a side (longitudinal) profile of a convex transverse section is taken, and a straight line S₁ parallel to the longitudinal axis of the horn is drawn to connect two endpoints (P₁, P₂) selected along its external axial curvature, all the points between the two endpoints will lie away from the line S₁ as compared to the centerline of the ultrasonic horn. In embodiments where only one convex transverse section is present, the maximum diameter of the convex transverse section typically is located distal to the halfpoint along the longitudinal length, that is, between the halfpoint and the output end. In some embodiments, the convex transverse section(s) can have a maximum diameter between about λ/4 and about λ/9. In certain embodiments, the convex transverse section(s) can include a maximum diameter greater than the diameter of the input surface.

In various embodiments, the present ultrasonic horns can include at least one concave transverse section. A concave transverse section can be defined by a narrow intermediate section between two wider ends. Or described differently with reference to FIG. 2, if a side profile of a concave transverse section is taken, and a straight line S₂ parallel to the longitudinal axis of the horn is drawn to connect two endpoints (P₃, P₄) selected along its external axial curvature, all the points between the two endpoints will lie between the line S₂ and the centerline of the ultrasonic horn. In embodiments where only one concave transverse section is present, the minimum diameter of the concave transverse section typically is located proximal to the halfpoint along the longitudinal length, that is, between the halfpoint and the input end. In some embodiments, the concave transverse section(s) can have a minimum diameter between about λ/8 and about λ/40. In certain embodiments, the concave transverse section(s) can include a minimum diameter less than the diameter of the output surface. In other words, the concave transverse section(s) can define a neck region having a minimum diameter along the entire longitudinal length of the present ultrasonic horns. In other embodiments, this neck region can have a diameter substantially similar to the diameter of the output surface.

In various embodiments, ultrasonic horns of the present teachings are adapted to transmit sound energy within a range of ultrasonic frequencies from about 15 kHz to about 30 kHz, for example, from about 15 kHz to about 20 kHz. In particular embodiments, the ultrasonic horns are adapted to resonate at about 18 kHz. The longitudinal length of the present ultrasonic horns is optimally chosen with reference to the wavelength of the ultrasonic vibrations. More specifically, the longitudinal length of the present ultrasonic horns is preferably selected to cause the horns to resonate at a selected frequency. Thus, once an ultrasonic frequency is selected, the corresponding wavelength of the vibrations in the material from which the horn is manufactured and hence the optimal longitudinal dimension of the horn can be determined. In various embodiments, the present ultrasonic horns can have a longitudinal length that approximates or is substantially equal to an integer multiple (n) of one-half of the corresponding wavelength (λ) of the resonant vibrations in the material from which the horn is manufactured. In certain embodiments, the present ultrasonic horns are half wavelength horns, meaning that the longitudinal length of these horns approximates n times (λ/2), where n is an odd integer greater than or equal to 1. In certain embodiments, the present ultrasonic horns are full wavelength horns, meaning that the longitudinal length of these horns approximates n times (λ/2), where n is an even integer greater than or equal to 2. The integer multiple n also corresponds to the number of nodes or nodal planes (at which the amplitude is zero) that a half wavelength horn or a full wavelength horn can have.

With most existing full-wavelength horn designs, the antinode, i.e., the point at which maximum displacement is observed, often is located within the horn and away from the output end. This is often the result of a compromise between gain and working surface area. With the present ultrasonic horns, the gain is optimized, and in various embodiments of full-wavelength horns, the maximum amplitude is observed (i.e., the antinode is located) at the output end. In certain embodiments, the output end is configured to reach a maximum displacement amplitude of up to about 150 μm.

In various embodiments, the present ultrasonic horns are made from materials of high fatigue strength that have a small density to modulus of elasticity ratio. In embodiments where the present ultrasonic horns are intended for treatment of corrosive liquids and heat-resistant properties are desired, a nickel-based alloy can be used. An example is an alloy known as NIMONIC 80A™ which contains about 70% nickel, about 18-21% chromium, about 1.8-2.7% titanium, about 1.0-1.8% aluminum, and about 2% cobalt. Other suitable materials include various metals and alloys, for example, steel, titanium alloys, or aluminum alloys. In some embodiments, a class of steels known as alloy tool steels, can be used. An example of an alloy tool steel is the alloy recognized in the steel industry as 2-A tooling steel, which is a fine-grain, air-hardened, steel containing about 0.95-1.24% carbon, about 4.75-5.50% chromium, about 0.90-1.4% molybdenum, about 0.15-0.50% vanadium, and about 1.00% manganese.

In certain embodiments, the present ultrasonic horns can be made of a nickel-chromium alloy that contains titanium and aluminum (e.g., NIMONIC 80A™) having a longitudinal length between about 300 mm and about 350 mm. Ultrasonic horns according to these embodiments can function as a full wavelength resonator at a resonant frequency of about 18 kHz.

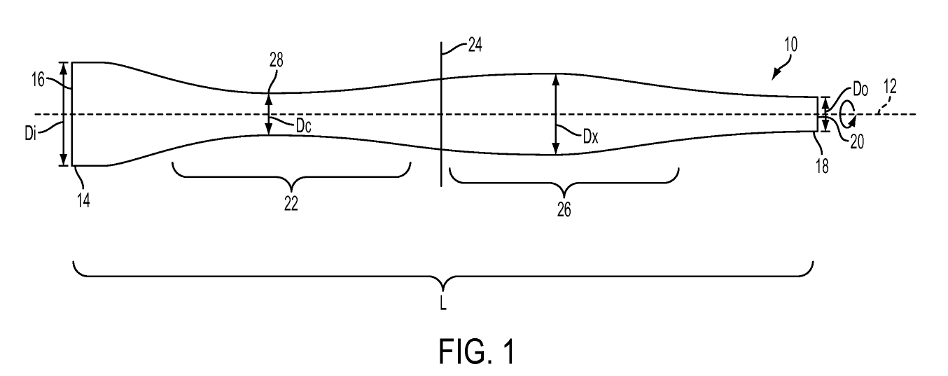

An embodiment of the present ultrasonic horns will be described with reference to FIG. 1, which shows the side view of an ultrasonic horn 10. The ultrasonic horn 10 is a solid elongated body that is symmetrical about the longitudinal axis 12. The ultrasonic horn 10 is defined by an input end 14 which includes the input surface 16, and an output end 18 which includes the output surface 20. The ratio of the diameter Dₒ of the output surface 18 to the diameter Dᵢ of the input surface 16 is about ⅓. For example, Dₒ can be about 15 mm (±2.5 mm), and Dᵢ can be 40 mm (±5 mm). As shown, the ultrasonic horn features a curvilinear profile including a concave transverse section 22 and a convex transverse section 26. The concave transverse section 22 includes a neck region 28 located between the input end 14 and the halfpoint 24 which has a diameter D_{c} substantially similar to the diameter of the output surface Dₒ. For example, D_{c} can be about 15-25 mm. The convex transverse section 24 can have a maximum diameter Dₓ located between the halfpoint 24 and the output end 18 that is substantially similar to the diameter of the input surface Dᵢ. For example, Dₓ can be about 30-40 mm. It can be seen from FIG. 1 that the diameter of the ultrasonic horn varies very gradually and continuously along most of its longitudinal length L, which can be between about 300 mm and about 360 mm.

FIG. 2 shows a side view of another embodiment of the present ultrasonic horns. Similar to the ultrasonic horn 10 in FIG. 1, the ultrasonic horn 110 also is a solid elongated body that is symmetrical about the longitudinal axis 112. The ultrasonic horn 110 is defined by an input end 114 which includes the input surface 116, and an output end 118 which includes the output surface 120. The ratio of the diameter Dₒ of the output surface 118 to the diameter Dᵢ of the input surface 116 is about 4/7 or about 0.57. For example, Dₒ can be about 45 mm (±5 mm), and Dᵢ can be 70 mm (±5 mm). Compared to the ultrasonic horn 10 in FIG. 1, the ultrasonic horn 110 has a more pronounced curvature. The concave transverse section 122 includes a very narrow neck region 128 which has a diameter D_{c} smaller than the diameter of the output surface Dₒ. For example, D_{c} can be about 20-30 mm. At the same time, the convex transverse section 126 has a maximum diameter Dₓ that is greater than the diameter of the input surface Dᵢ. For example, Dₓ can be about 65-80 mm. The longitudinal length L of the ultrasonic horn can be between about 300 mm and about 360 mm.

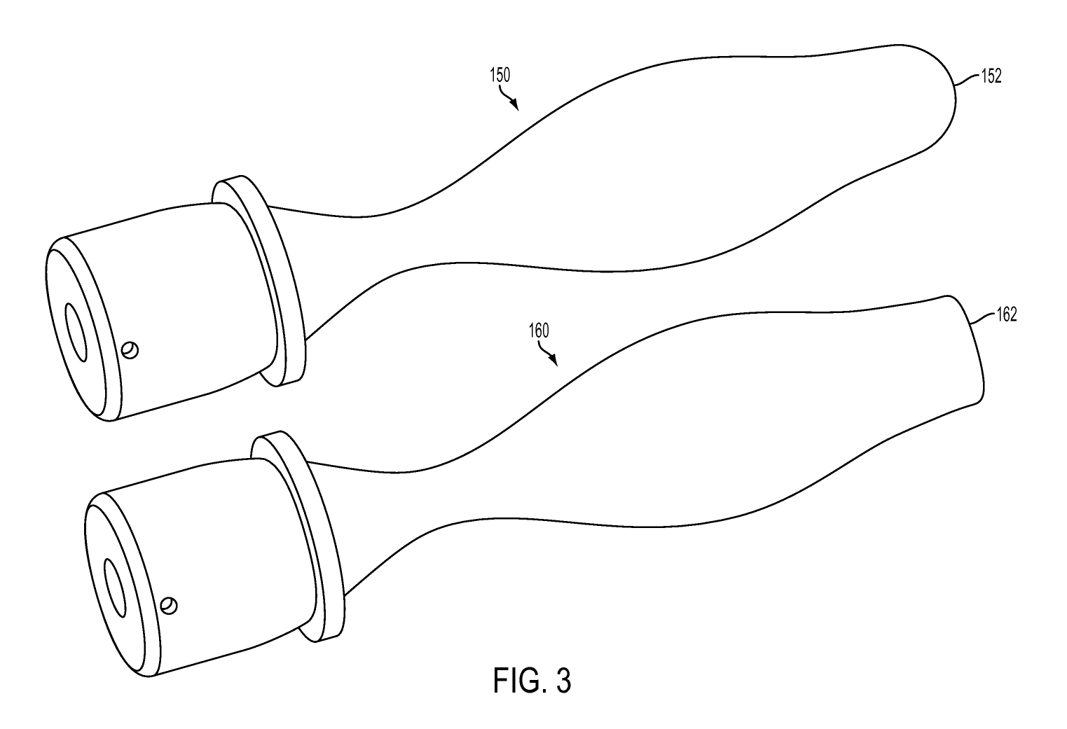

FIG. 3 shows two ultrasonic horns 150 and 160 according to the present teachings that are identical except for the shape of their respective output ends. As shown, horn 160 has a flat output surface 162, whereas horn 150 has a rounded output end 152 that is spherically shaped. Compared to a flat output surface, a rounded output end can change the shape of the cavitation zone in a liquid and improve the flow of the liquid in a typical flow-through reactor setup. Data obtained from finite element analysis (FEA) suggest that the rounding of the output end has little impact on stress distribution when compared to an identically shaped horn with a flat output surface.

The present ultrasonic horns can be machined as a solid continuous piece. By “continuous,” it is meant that the body does not contain internal cavities, but instead is fully dense according to its external dimensions. In some embodiments, the solid elongated body of the present ultrasonic horns can be of unitary construction, which means that it is formed from a single piece of material, rather than from multiple pieces or components that are formed individually and then joined by welding, or by the use of bolts, clamps, or any other method of securing parts together. Unitary construction of the present ultrasonic horns can be achieved by any conventional method of forming metal or alloy parts. Examples of these methods include conventional machining, forging, and casting. Generally, any method that will not compromise the grain structure or the strength of any portion of the horn can be used. In other embodiments, the elongated body can be composed of multiple parts that are rigidly joined to each other, using methods that minimize undesirable freedom of movement and/or scattering, for example, by friction welding or electron beam welding as known by those skilled in the art.

For embodiments intended for treating corrosive liquids, the present ultrasonic horns either can be clad with a corrosion-resistant material or left without cladding. Embodiments that are clad can be either clad in their entirety or clad only on the portions that will be in contact with the corrosive fluid reaction medium. Examples of suitable cladding materials include silver-based metals, including both silver itself and alloys in which silver is the major component. Alloys in which silver constitutes 85% or more by weight or preferably 90% or more by weight can be used, with alloying components selected from one or more of copper, zinc, and cadmium.

The present ultrasonic horns can be incorporated into a system (e.g., a reactor) for ultrasonic treatment of liquids. The present ultrasonic horns can be mounted to a reaction vessel, where at least the output end of the ultrasonic horn is positioned inside the vessel. The ultrasonic horn can be submerged in the vessel to various portion of its length as needed to optimize cavitation of the target liquid. The input end of the ultrasonic horn is in operative contact with a source of ultrasonic energy. In some embodiments, the input end is directly coupled to an ultrasonic transducer. In some embodiments, the ultrasonic transducer can be attached (e.g., by welding) to a resonator that provides a slight pre-amplification. For example, the resonator can be a half wavelength cylinder. The transducer, or the transducer/resonator combination can be attached to the ultrasonic horn by various mechanical means, e.g., by bolting the transducer, transducer/resonator combination to the ultrasonic horn by a stud or similar means. For mounting to the reaction vessel, the present ultrasonic horns can contain a mounting fixture, such as, for example, a flange, a shoulder, an extension, bolt holes, and the like. In certain embodiments, the mounting fixture can be positioned at or near a node. For example, the ultrasonic horn can be mounted to the reaction vessel along its proximal nodal plane (near the input end), along its distal nodal plane (near the output end), and/or slightly proximal to the distal nodal plane where the surface normal displacement is zero.

In certain embodiments, the mounting fixture can allow the input end and the ultrasonic transducer that is coupled to the input end to be surrounded by a coolant jacket. Coolant can be circulated through the jacket in these embodiments to control the temperature rise caused by the electro-mechanical efficiency of the transducer and the input end. An ultrasonic horn according to the present teachings also can include o-rings, gaskets, or the like to form seals around its elongated body at locations where the ultrasonic horn enters the reaction chamber, the coolant jacket, or both.

Any of a wide variety of ultrasonic transducers can be used for producing ultrasonic vibrations in the ultrasonic horn. Any of mechanical, electrical, electromagnetic, or thermal energy sources can be used to generate ultrasonic waves, although the intensity level that can be attained by different types of sources tends to vary. A typical electromagnetic source is a magnetostrictive transducer which converts magnetic energy into ultrasonic energy by applying a strong alternating magnetic field to certain metals, alloys and ferrites. A typical electrical source is a piezoelectric transducer, which uses natural or synthetic single crystals (such as quartz) or ceramics (such as barium titanate or lead zirconate) and applies an alternating electrical voltage across opposite faces of the crystal or ceramic to cause an alternating expansion and contraction of crystal or ceramic at the desired frequency.

For high-energy applications, the ultrasonic transducer can be a loop-shaped transducer that converts periodically varying voltages to mechanical vibrations in the ultrasound range by way of magnetostriction. The loop can be formed as a stack (or multiple stacks) of thin, flat plates of magnetostrictive material laminated together with dielectric material such as an oxide layer, a plastic resin or a ceramic adhesive between each pair of adjacent plates. The number of plates in the stack can range from 100 to 400 or more plates, and the thickness of each plate can range from about 50 microns to about 250 microns.

The size of each plate and hence the loop can vary. In various embodiments, each plate can have a length ranging from about 5 cm to about 30 cm (e.g., about 13 cm), and a lesser width, generally ranging from about 3 cm to about 10 cm (e.g., about 6 cm). The central opening of the loop will typically range from about 0.5 cm to about 5 cm. The transducer loop typically is wound with a coil of electrically conductive wire (i.e., a drive coil), and the windings are arranged and oriented to produce magnetostrictive vibrations in the loop when a varying voltage is imposed across the windings. The windings, for example, can be coiled in one direction around one lengthwise side of the loop and in the opposite direction around the other lengthwise side.

The transducer can be powered by any oscillating voltage. The oscillations can assume any waveform, ranging for example, from a sinusoidal waveform to a rectangular waveform. The voltage amplitudes can be from about 140 volts to about 300 volts. The wattages can be from about 2 kilowatts to about 10 kilowatts. The frequency of the voltage oscillation will be selected to achieve the desired ultrasound frequency, for example, in the range of about 10 kHz to about 30 kHz, including from about 15 kHz to about 20 kHz. Examples of ultrasonic transducers are described in U.S. Pat. Nos. 6,897,628 and 7,275,440, the disclosure of each of which is incorporated by reference herein in its entirety.

Ultrasonic horns according to the present teachings can be used in either batch reactors to promote batch-wise reactions or in continuous-flow reactors for reactions performed in a continuous manner. When the present ultrasonic horns are used in continuous flow-through reactors, the flowing reaction medium will provide cooling of the ultrasonic horn at the output end. It can be beneficial to cool the input end of the ultrasonic horn and the ultrasound transducer, using a cooling system that is independent of the reaction medium. Cooling of the input end of the ultrasonic horn and the ultrasound transducer can be conveniently achieved by enclosing these loops in a jacket or housing through which a coolant is passed or circulated. The ultrasonic horn can be equipped with a secondary mounting fixture so that its input end and the transducer can be enclosed in the jacket in a fluid-tight manner. For example, oil or water can be used as an effective and convenient coolant medium for circulation through the jacket.

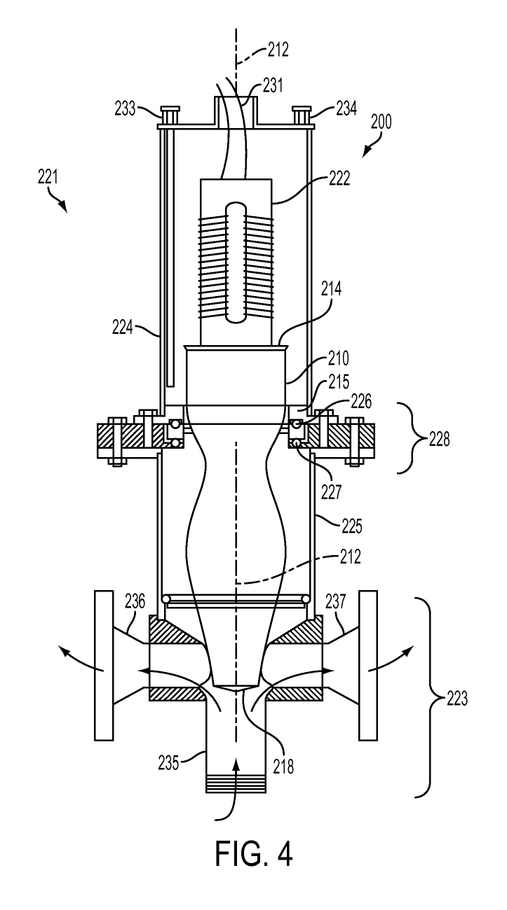

FIG. 4 is a cross-section of a flow-through reactor 200, which includes a coolant chamber assembly 221 inside of which are an ultrasonic horn 210 according to the present teachings and an ultrasound transducer 222. A coolant jacket 224 surrounds the ultrasonic transducer 222 and the input end 214 of the ultrasonic horn. The reactor also includes a continuous flow-through reaction chamber 223 and a connecting cylinder 225, which is an extension of the reaction chamber 223 and in operation contains neither the reaction medium nor the coolant, that joins the reaction chamber 223 to the coolant jacket 224. The coolant jacket 224 is closed at the bottom by the mounting flange 215 and sealed with o-rings 226, 227 at the flange 215. The coolant jacket 224, the reaction chamber extension 223, and the horn 210 are secured together by an arrangement of flanges and bolts 228 at the level of the mounting flange 215 of the ultrasonic horn. Electric leads 231 connect the ultrasound transducer 222 to an external power supply, amplifier and controller (not shown). A coolant inlet 233 directs coolant to the jacket interior and the heated coolant leaves through a coolant outlet 234. More details about flow-through reactors can be found in U.S. Patent Publication No. US 2005/0274600, the disclosure of which is incorporated by reference herein in its entirety.

The reaction medium that is treated with ultrasound enters the reaction chamber 223 through an inlet port 235 which is coaxial with the longitudinal axis 212 of the ultrasonic horn, and leaves the reactor through exit ports 236, 237 laterally positioned on the sides of the reaction chamber. The output end 218 of the ultrasonic horn is positioned directly in the mouth of the inlet port 235 so that the incoming reaction medium strikes the distal end 218, flows radially outward over the surface of the output end 218 and leaves through the exit ports 236, 237.

The power components, including the power supply, the amplifier, and the controller, are conventional components available from commercial suppliers and readily adaptable to perform the functions described above. A computer-controlled arbitrary waveform generator such as the Agilent 33220A or Advantek 712 with an output DAC (digital-to-analog converter) or a microprocessor-drive, voltage-controlled waveform generator designed from an 8038 integrated circuit chip can be used. The arbitrary waveform generator can be auto-tuned by an output DAC on a microprocessor or by functions in a LabVIEW™ computer (National Instruments Corporation, Austin, Tex., USA), in which pulse software controls the arbitrary waveform generator to maximize the ultrasonic output by adjusting the pulse frequency to the transducer resonance frequency. The positive and negative pulse components also can be adjusted to give an overall DC component that will maximize the magnetostrictive effect.

Integrated gate bipolar transistors in a full bridge power configuration can be used as power components. One such configuration is a full bridge power configuration using four integrated gate bipolar transistors (IGBTs) formed in a configuration of two half-bridge push-pull amplifiers. Each half bridge section is driven by an asymmetrical rectangular pulse train, the trains being 180 degrees out of phase. The relative amounts of the positive and negative pulse components that drive each half bridge section can be optimized for maximum ultrasound output power. Each IGBT is isolated from the signal source by an opto-isolation driving transistor. An ultrasound generator that can be used in accordance with the present teachings is described in U.S. Pat. No. 7,408,290, the disclosure of which is incorporated by reference herein in its entirety.

Accordingly another aspect of the present teachings relates to a process of treating a liquid composition (e.g., a liquid reaction medium) with ultrasonic vibrations. The process can be performed either in a batchwise manner or in a continuous-flow operation, for example, using the continuous flow-through reactor described herein. The liquid composition can be an aqueous emulsion, for example, an emulsion including 90% crude oil and 10% water (by volume). The process can include contacting at least the output end of an ultrasonic horn according to the present teachings with a liquid composition; delivering ultrasonic vibrations to the input surface of the ultrasonic horn; amplifying the ultrasonic vibrations through the solid elongated body of the ultrasonic horn; transmitting the ultrasonic vibrations to the liquid composition to cause agitation of the liquid composition. The liquid composition can be contained in a reaction vessel, where the reaction vessel has an entry port and an exit port arranged such that as the liquid composition enters the reaction vessel through the entry port, it contacts and flows across at least the output surface of the ultrasonic horn before leaving the reaction vessel through the exit port. Therefore, the present process can include introducing the liquid composition into the reaction vessel through the entry port; flowing the liquid composition across the output end including the output surface of the ultrasonic horn; and removing the liquid composition from the reaction vessel through the exit port. More specifically, the incoming aqueous emulsion can impinge the output surface of the ultrasonic horn then flow radially outward to the edges of the output surface and along the sides (circumferential surfaces) of the ultrasonic horn before leaving the reactor. The process can involve passing the liquid composition into and out of the reaction vessel at a rate between about 0.5 L/s and about 10 L/s.

The present ultrasonic horns and related systems can be useful in the performance of any chemical reaction whose yield, reaction rate, or both can be enhanced by ultrasonic treatment. For example, the present ultrasonic horns and related systems can be used in the desulfurization of liquid fossil fuels including crude oil and crude oil fractions. Processes disclosing the use of ultrasound in treating these materials are described in for example, U.S. Pat. Nos. 6,402,939, 6,500,219, 6,652,992, and 6,827,844, and 7,300,566, and U.S. Patent Publication No. US 2003-0051988, the disclosure of each of which is incorporated by reference herein in its entirety.

As used herein, “liquid fossil fuel” refers to any carbonaceous liquid that is derived from crude oil (or petroleum), coal, or any other naturally occurring material and that is used for energy generation. Included among these fuels are automotive fuels such as gasoline, diesel fuel, jet fuel, and rocket fuel. In particular embodiments, the reaction medium can be a residual carbonaceous liquid that is derived from crude oil (or petroleum), coal, or any other naturally occurring material. Petroleum residuum-based fuel oils (or “resids”) are the heavy fraction remaining after petroleum crudes are distilled at atmospheric pressure or at reduced pressure, i.e., the residue left after the most readily accessible components of the petroleum are extracted. Resids are highly complex in composition, including components of high molecular weight as well as polynuclear aromatics, coke, asphaltenes, resins, small ring aromatics, and saturates. Petroleum residua and residuum-based fuel oils, including bunker fuels and residual fuels, are of particular interest. No. 6 fuel oil, for example, which is also known as “Bunker C” fuel oil, is used in oil-fired power plants as the major fuel and is also used as a main propulsion fuel in deep draft vessels in the shipping industry. No. 4 fuel oil and No. 5 fuel oil are used to heat large buildings such as schools, apartment buildings and office buildings, and large stationary marine engines. The heaviest fractions are resids, including the vacuum residuum from the fractional distillation, commonly referred to as “vacuum resid,” with a boiling point of 565° C. and above, which is used as asphalt and coker feed. The present teachings can be used to treat any of these oils or fractions for purposes of increasing the proportion of usable oils and other petroleum products that can be extracted from them.

Depending on the type of fuel being treated, the duration and the amount of the exposure of the liquid composition to ultrasound in accordance with the present teachings can vary. Generally, effective and useful results can be achieved in accordance with the present teachings with ultrasonic energy exposure of a relatively short period of time, notably less than twenty minutes and in many cases less than ten minutes. The ultrasonic energy can be applied to the liquid composition in a batchwise manner or in a continuous manner in which case the exposure time is the residence time in a flow-through reactor.

While not intending to be bound by any particular theory, it has been reported that the application of ultrasound to a liquid composition produces cavitation in the liquid, i.e., the continuous formation and collapse of microscopic vacuum bubbles with extremely high localized temperatures and pressures. For example, it is believed that ultrasonic waves at a frequency of 45 kHz produce 90,000 formation-implosion sequences per second and localized temperatures on the order of 5,000° C. and pressures on the order of 4,500 psi. This causes extreme turbulence and intense mixing.

Accordingly, in various embodiments, the present process can include applying ultrasonic vibrations to a liquid reaction medium including a fossil fuel for a time sufficient to cause oxidation of sulfides in the fossil fuel to sulfones, and extracting the sulfones to yield an organic phase that is substantially sulfone-free. This can include applying ultrasonic vibrations at a frequency of from about 15 kHz to about 200 kHz (e.g., from about 15 kHz to about 50 kHz) and an intensity of from about 30 watts/cm² to about 300 watts/cm² (e.g., from about 50 watts/cm² to about 100 watts/cm²).

Certain organic sulfur compounds that are typically present in fossil fuels can be illustrative of the effectiveness of the process. These compounds include dibenzothiophene and related sulfur-bearing organic sulfides. Ultrasound-promoted oxidation has been shown to be selective toward the sulfur-bearing compounds of the fossil fuel, with little or no oxidative effect in the non-sulfur bearing components of the fuel. In embodiments where a continuous flow-through set-up is used, a large quantity of fossil fuel can be treated at a modest operating cost and a low residence time in the reaction chamber. For example, residence time in the reaction chamber can be from about 0.5 minute and about 30 minutes. In some embodiments, the residence time can be between about 0.3 minute and about 5 minutes.

The present ultrasonic horns and related systems can be useful in other applications which ultrasound has been used. These other applications include cleaning for the electronics, automotive, aircraft, and precision instruments industries, flow metering for closed systems such as coolants in nuclear power plants or for blood flow in the vascular system, materials testing, machining, soldering and welding, electronics, agriculture, oceanography, and medical imaging.

The following examples are provided to illustrate further and to facilitate the understanding of the present teachings and are not in any way intended to limit the invention.

### Examples

A first ultrasonic horn (Horn 1) according to the shape and form shown in FIG. 1 was made as a solid unitary piece from NIMONIC 80A™, with the following dimensions: L=300 mm (±3 mm); Dᵢ=40 mm (±1 mm); Dₒ=15 mm (±1 mm); D_{c}=13 (±1 mm); and Dₓ=31 mm (±1 mm).

A second ultrasonic horn (Horn 2) according to the shape and form shown in FIG. 2 was made as a solid unitary piece from NIMONIC 80A™, with the following dimensions: L=340 mm (±5 mm); Dᵢ=75 mm (±2 mm); Dₒ=46 mm (±1 mm); D_{c}=20 mm (±2 mm); and Dₓ=74 mm (±1 mm).

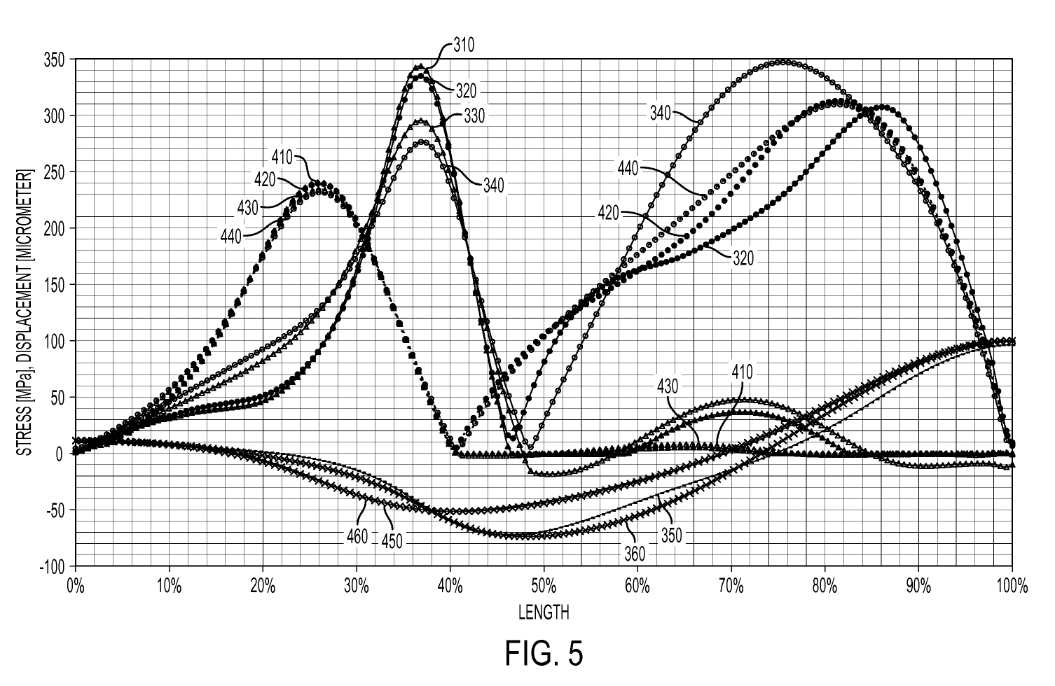

Both horns were operated at a frequency of about 18 (+0/−0.05) kHz; and the gain was measured to be about 9 (−0/+0.1). FIG. 5 compares the stress profiles and the displacement profiles of Horn 1 and Horn 2 as analyzed by Finite Element Analysis (FEA) along their longitudinal lengths. The horn lengths are normalized to represent the input end as 0% and the output end as 100%. The stress profiles include principal stress, von Mises stress as analyzed by FEA along the surface and the centerline. The stresses are normalized for 100 micron of amplitude at the output end. The displacement profiles also were analyzed by FEA both along the surface and the centerline. The following notations are used:

| Profile | Description |
|---|---|
| 310 (solid line, solid triangle) | Horn 1, principal stress, along surface |
| 320 (solid line, solid circle) | Horn 1, von Mises stress, along surface |
| 330 (solid line, open triangle) | Horn 1, principal stress, along center- |
| | line |
| 340 (solid line, open circle) | Horn 1, von Mises stress, along center- |
| | line |
| 350 (solid line, rectangle) | Horn 1, displacement, along surface |
| 360 (solid line, cross) | Horn 1, displacement, along centerline |
| 410 (dotted line, solid triangle) | Horn 2, principal stress, along surface |
| 420 (dotted line, solid circle) | Horn 2, von Mises stress, along surface |
| 430 (dotted line, open triangle) | Horn 2, principal stress, along center- |
| | line |
| 440 (dotted line, open circle) | Horn 2, von Mises stress, along center- |
| | line |
| 450 (dotted line, rectangle) | Horn 2, displacement, along surface |
| 460 (dotted line, cross) | Horn 2, displacement, along centerline |

Referring to FIG. 5, it can be seen for both Horns 1 and 2, that the maximum amplitude was observed when L is 100%, i.e., at the output end. For ease of comparison, the maximum amplitude at the output end has been normalized to 100%. When L is 50% (i.e., at the halfpoint), the amplitude is about 50-80% of the maximum amplitude at the output end. The nodes for Horn 1 are located at L˜20% and L˜75%. The nodes for Horn 2 are located at L˜15% and L˜70%.

With continued reference to FIG. 5, it can be seen that both ultrasonic horns have well-distributed stress along the length of the horn. With about 100 micron of displacement at the output end, the maximum von Mises stress along the surface or the centerline was analyzed to be less than about 350 MPa.

### Other Embodiments

The present teachings can be embodied in other specific forms, not delineated above, without departing from the spirit or essential characteristics thereof. The foregoing embodiments are therefore to be considered in all respects illustrative rather than limiting on the present teachings described herein. Scope of the present invention is thus indicated by the appended claims rather than by the foregoing description, and all changes that come within the meaning and range of equivalency of the claims are intended to be embraced therein.

What is claimed is:

1. An ultrasonic horn comprising a solid elongated body adapted to transmit ultrasonic vibrations from an input end defining an input surface to an output end defining an output surface, wherein:
the solid elongated body comprises a material and a longitudinal length extending between the input end and the output end, the longitudinal length approximating an integer multiple of one-half of a wavelength (X) of a resonant frequency of the material; and
the solid elongated body comprises a plurality of transverse sections along the longitudinal length, the plurality of transverse sections comprising a first transverse section having a convex longitudinal profile.

2. The ultrasonic horn of claim 1, wherein the first transverse section has a maximum diameter greater than the diameter of the input end.

3. The ultrasonic horn of claim 1, wherein the maximum diameter of the first transverse section is located between the output end and a half-point of the longitudinal length.

4. The ultrasonic horn of claim 1, wherein the plurality of transverse sections comprise a second transverse section having a concave longitudinal profile.

5. The ultrasonic horn of claim 4, wherein the minimum diameter of the second transverse section is located between the input end and a half-point of the longitudinal length.

6. The ultrasonic horn of claim 4, wherein the minimum diameter of the second transverse section is less than the diameter of the output end.

7. The ultrasonic horn of claim 1, wherein the output end comprises a flat output surface.

8. The ultrasonic horn of claim 1, wherein the output end comprises a rounded tip.

9. The ultrasonic horn of claim 1, wherein the solid elongated body is constructed from a unitary piece of material selected from a corrosive-resistant metal or a corrosive-resistant alloy.

10. The ultrasonic horn of claim 1, wherein the material is a nickel-chromium alloy comprising titanium and aluminum.

11. The ultrasonic horn of claim 1, wherein the ratio of the diameter of the output surface to the diameter of the input surface is greater than about ⅓.

12. The ultrasonic horn of claim 1, wherein the output end is configured to reach a maximum displacement amplitude of up to about 150 μm.

13. The ultrasonic horn of claim 1, wherein the ultrasonic horn is configured to exhibit, at a halfpoint along the longitudinal length, a displacement amplitude that is equivalent to about 50-80% of the maximum displacement amplitude located at the output end.

14. The ultrasonic horn of claim 1, wherein the ultrasonic horn is configured to provide a gain of about 8.0 or greater.

15. The ultrasonic horn of claim 1, wherein the ultrasonic horn is configured to exhibit a maximum von Mises stress lower than about 400 MPa per 100 micron of displacement at the output end.

16. A flow-through reactor for the treatment of a liquid composition with ultrasonic energy, the flow-through reactor comprising:
a reaction vessel defining a reaction chamber; and
the ultrasonic horn of claim 1, wherein the ultrasonic horn is mounted to the reaction vessel with at least the output end extending into the reaction chamber.

17. The flow-through reactor of claim 16, wherein the reaction vessel comprises an entry port and an exit port arranged to cause a liquid composition entering the reaction chamber through the entry port to contact and flow across at least the output surface of the ultrasonic horn before leaving the reaction chamber through the exit port.

18. A process for treating a liquid composition with ultrasonic vibrations, the process comprising:
delivering ultrasonic vibrations to the input surface of the ultrasonic horn of claim 1;
amplifying the ultrasonic vibrations through the solid elongated body of the ultrasonic horn; and
transmitting ultrasonic vibrations from the output surface of the ultrasonic horn to a liquid composition.

19. The process of claim 18, comprising passing the liquid composition at a rate between about 0.5 L/s and about 10 L/s.

20. The process of claim 18, wherein the liquid composition comprises fossil fuel.
